(19) United States
(12) Patent Application Publication
(10) Pub. No.: US 2025/0281223 A1
(43) Pub. Date: Sep. 11, 2025

(54) Systems and Methods for Using Cortical Lesioning to Treat Conditions

(71) Applicant: Cadwell Laboratories, Inc., Kennewick, WA (US)

(72) Inventor: John A. Cadwell, Richland, WA (US)

(21) Appl. No.: 19/073,118

(22) Filed: Mar. 7, 2025

Related U.S. Application Data
(60) Provisional application No. 63/562,391, filed on Mar. 7, 2024.

(51) Int. Cl. A61B 18/08; A61B 18/00

(52) U.S. Cl. CPC A61B 18/082; A61B 2018/00446; A61B 2018/00577; A61B 2018/00648; A61B 2018/0072; A61B 2018/00791; A61B 2018/00839

(57) ABSTRACT

A system for treating epileptic seizures by lesioning an affected area of a patient's brain is provided. The system includes at least one depth electrode comprising one or more contacts and one or more heating modules positioned longitudinally along a shaft. The depth electrode is insertable in the affected area of the patient's brain, at least one of the contacts is used to monitor EEG activity of the brain, and a heat controller coupled with the shaft for providing an energy input to one or both of the contacts or the heating modules for generating a lesion in the affected area of the patient's brain based on the concurrently and continuously monitored EEG activity of the brain.

### CROSS-REFERENCE

The present application relies on, for priority, U.S. Patent Provisional Application No. 63/562,391, titled “Systems and Methods for Using Cortical Lesioning to Treat Conditions” and filed on Mar. 7, 2024, which is herein incorporated by reference in its entirety.

### FIELD

The present specification relates to systems for cortical lesioning of an affected area of a patient's brain using heat while continuously monitoring the EEG activity of the brain. More specifically, the present specification relates to the use of cortical lesioning for the mitigation of an epileptic condition.

### BACKGROUND

Epilepsy is a disorder of the brain characterized by abnormal electrical activity in the brain that results in repeated seizures. A seizure is usually defined as a sudden alteration of behavior due to a temporary change in the electrical functioning of the brain. With epilepsy, the brain's electrical rhythms become imbalanced, resulting in recurrent seizures. In patients with seizures, the normal electrical pattern is disrupted by sudden and synchronized bursts of electrical energy, that may begin in one small area of the brain and spread to connected areas, and that may briefly affect their consciousness, movements or sensations. The seizure activation sites may be focal, i.e. starting in only one location, or may be multifocal. The symptoms experienced by patients from seizure activity vary from absence attacks, auras, sensory, or convulsive motor phenomenon which can be limited or generalized.

A first-line of treatment usually involves using anti-epileptic drugs to inhibit the seizure focus, which is the site in a patient's brain from which the seizure originated. If drug therapy is ineffective, surgical procedures such as cortical lesioning may be considered for removing the seizure focus. Usually, in cases of patients that are resistant to anti-epileptic drugs and are candidates for the surgical procedures, it is advantageous to provide an early treatment as continuing seizures progressively damage the brain.

The goal of a cortical lesioning procedure is to achieve better seizure control without causing loss of brain function, while minimizing damage to surrounding normal tissue. The surgical procedure can be performed by surgical excision, laser ablation or electro-cauterization. While performing surgical lesioning/excision, limiting a size of the lesion while targeting abnormal brain tissue and leaving normal tissue undamaged, is desired. Usually, such localization is performed using electroencephalogram (EEG) which involves placing electrodes in or near the suspected seizure focus and watching or provoking epileptiform activity via the electrodes, for determining which electrodes are closest to the abnormal activity. One such known method involves placing a grid electrode on the brain's surface through open craniotomy, monitoring EEG activity for several days, and then performing a second surgery to remove the grid electrode and excising abnormal brain tissue. Since the cortical lesioning procedure described above is surgical and invasive (requires open craniotomy) and may have severe complications leading to unintended brain damage, patients/doctors tend to defer this type of treatment for as long as possible or not use it altogether.

Hence, there is need for a minimally invasive cortical lesioning procedure for treatment of epilepsy that is safer to conduct and may be provided at an early stage of the disease. There is also a need for a method that enables both monitoring and ablating the seizure focus in a single surgical process.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification is directed towards a cortical lesioning system comprising: at least one depth electrode comprising a shaft having a length defined by a longitudinal axis; one or more contacts positioned on the shaft and spaced apart along said longitudinal axis; and one or more heating modules positioned on the shaft and spaced apart along said longitudinal axis, wherein the depth electrode is configured to be insertable in a target area of a patient's brain and wherein at least one of the one or more contacts is adapted to be used to monitor EEG activity of the patient's brain; at least one heat controller coupled with the shaft for providing an electrical current to at least one of the one or more contacts or of the one or more heating modules for generating one or more lesions in the target area of the patient's brain; and an EEG recorder configured to capture and generate an EEG output using data from said at least one depth electrode, wherein said EEG recorder is configured to capture and generate said EEG output a) while the at least one heat controller is providing said electrical current to the at least one of the one or more contacts or of the one or more heating modules or b) only when the at least one heat controller is not providing said electrical current to the at least one of the one or more contacts or of the one or more heating modules.

Optionally, the at least one depth electrode is adapted to be inserted at a predefined angle and depth in the vicinity of a seizure focus in the patient's brain.

Optionally, the heat controller is further configured to generate said electrical current based upon a lesioning sequence, and wherein said lesioning sequence defines a pattern for the one or more lesions.

Optionally, the system further comprises an EEG controller, wherein the EEG controller comprises the EEG recorder and an EEG lesioning software application, wherein, when executed, the EEG lesioning software application generates said lesioning sequence and communicates the lesioning sequence to the heat controller. Optionally, the EEG controller further comprises the heat controller.

Optionally, the heat controller comprises a switch matrix configured to couple at least one of the one or more contacts or the one or more heating modules to at least one of an AC power source or a DC power source.

Optionally, the shaft is coupled to the heat controller via a lead wire.

Optionally, when executed, the EEG lesioning software application is further adapted to generate a virtual model of the at least one depth electrode and wherein the EEG lesioning software application is configured to generate the virtual model by using trajectory information indicative of a placement of the at least one depth electrode in the target area of the patient's brain.

Optionally, the virtual model is configured to depict a 3D volumetric image comprising data representative of at least one of the patient's brain, a cortical surface of the brain, the one or more lesions, and an actual temperature in the brain.

Optionally, the one or more contacts are spaced along the longitudinal axis such that adjacent ones of the one or more contacts are separated from each other by a distance in a range of 2.5 mm to 10 mm.

Optionally, the one or more contacts are configured to only monitor electrical activity of the patient's brain and are not configured to receive the electrical current from the heat controller to generate heat.

Optionally, the one or more contacts are configured to monitor electrical activity of the patient's brain and to receive the electrical current from the heat controller to generate heat.

Optionally, the one or more heat modules are spaced along the longitudinal axis such that adjacent ones of the one or more heat modules are separated from each other by a distance less than 5 mm.

Optionally, the one or more contacts are configured to only monitor electrical activity of the patient's brain and are not configured to receive the electrical current from the heat controller to generate heat while the one or more heat modules are configured to receive the electrical current from the heat controller to generate heat.

In some embodiments, the present specification is directed towards a method of lesioning portions of a patient's brain, comprising: providing at least one depth electrode, wherein the at least one depth electrode comprises a shaft having a length defined by a longitudinal axis, one or more contacts positioned on the shaft and spaced apart along said longitudinal axis, and one or more heating modules positioned on the shaft and spaced apart along said longitudinal axis, wherein the depth electrode is configured to be insertable in a target area of a patient's brain and wherein at least one of the one or more contacts is adapted to be used to monitor EEG activity of the patient's brain; inserting the at least one depth electrode in a target area of the patient's brain; generating at least one lesion in the target area of the patient's brain by providing an electrical current to at least one of the one or more contacts or of the one or more heating modules in order to generate heat from the at least one of the one or more contacts or of the one or more heating modules; and generating an EEG output using data from said at least one depth electrode, wherein said EEG output is generated a) said electrical current is being provided to the at least one of the one or more contacts or of the one or more heating modules or b) only when said electrical current is not being provided to the at least one of the one or more contacts or of the one or more heating modules.

Optionally, the method further comprises inserting the at least one depth electrode at a predefined angle and depth in the vicinity of a seizure focus in the patient's brain.

Optionally, the method further comprises generating the electrical current based on a lesioning sequence generated by a lesioning software application.

Optionally, the method further comprises heating the seizure focus to a temperature of 160 degrees F.

Optionally, the method further comprises using data indicative of a trajectory of placement of the at least one depth electrode in the target area of the patient's brain to generate a virtual model of the at least one depth electrode.

Optionally, the method further comprises generating a 3D volumetric image of the patient's brain, a cortical surface presentation of the brain, the one or more lesions, and a temperature in the brain.

Optionally, the one or more contacts are spaced along the longitudinal axis such that adjacent ones of the one or more contacts are separated from each other by a distance in a range of 2.5 mm to 10 mm.

Optionally, the one or more contacts are configured to only monitor electrical activity of the patient's brain and are not configured to receive the electrical current to generate heat.

Optionally, the one or more contacts are configured to monitor electrical activity of the patient's brain and to receive the electrical current to generate heat.

Optionally, the one or more heat modules are spaced along the longitudinal axis such that adjacent ones of the one or more heat modules are separated from each other by a distance less than 5 mm.

Optionally, the one or more contacts are configured to only monitor electrical activity of the patient's brain and are not configured to receive the electrical current to generate heat while the one or more heat modules are configured to receive the electrical current to generate heat.

In some embodiments, the present specification discloses a cortical lesioning system comprising: at least one depth electrode comprising one or more contacts and one or more heating modules positioned on a shaft, wherein the depth electrode is configured to be insertable in a target area of a patient's brain, and wherein at least one of the contacts is used to monitor EEG activity of the brain; at least one heat controller coupled with the shaft for providing an electrical current to at least one of the contacts or the heating modules for generating one or more lesions in the target area of the patient's brain; and an EEG recorder to capture and generate an EEG output (including data, charts, tables, traces, images, or other indices of brain activity) using data from said at least one depth electrode, wherein said EEG recorder is configured to capture and generate said EEG output, under the control of an EEG controller, while the at least one heat controller is providing said electrical current or in between time periods within which the at least one heat controller is providing said electrical current (i.e. when the heat controller is not providing said electrical current).

Optionally, the system further includes an EEG lesioning software application coupled with the heat controller and configured to generate a lesioning sequence, wherein said lesioning sequence defines a pattern for generating the one or more lesions.

Optionally, the system further includes an EEG recorder coupled with the heat controller and adapted to record EEG activity of the patient's brain.

In some embodiments, the present specification discloses a method of lesioning portions of a patient's brain, comprising: providing at least one depth electrode having a plurality of contacts and a plurality of heating modules positioned on a shaft; inserting the depth electrode in a target area of the patient's brain; generating a lesion in the target area of the patient's brain by generating heat from at least one of the plurality of contact or the plurality of heating modules; and concurrent to generating heat, monitoring EEG activity of the brain using at least some of the plurality of contacts.

Optionally, the monitoring is performed using an EEG recorder adapted to capture and generate EEG output using data from said at least one depth electrode.

Optionally, the generating the lesion is performed using at least one heat controller coupled with the shaft and wherein the heat controller is configured to provide an electrical current to at least one of the plurality of contacts or the plurality of heating modules.

Optionally, the method further comprises inserting the at least one depth electrode at a predefined angle and depth in the vicinity of a seizure focus in the patient's brain.

Optionally, the method further comprises using MRI and EEG images of the patient's brain to determine the seizure focus and determine one or more lesions to be generated.

Optionally, the method further comprises using an EEG lesioning software application coupled with the heat controller to generate a lesioning sequence based on the one or more lesions to be generated.

Optionally, the method further comprises connecting at least one of the plurality of contacts and the plurality of heating modules to one of an AC power source or a DC power source.

Optionally, the method further comprises coupling the shaft to the heat controller using a lead wire.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a depth electrode comprising multiple contacts and heating modules, in accordance with an embodiment of the present specification;

FIG. 2A is a diagrammatic representation of a system for using cortical lesioning to treat epileptic seizures, in accordance with an embodiment of the present specification;

FIG. 2B illustrates virtual representations of a computed lesion and an adjusted lesion which is generated by a virtual lesion generator for viewing in a lesion viewer, in accordance with some embodiments of the present specification;

FIG. 3 is a diagrammatic representation of a switch matrix for use in a system for using cortical lesioning to treat epileptic seizures, in accordance with an embodiment of the present specification; and

FIG. 4 is a flowchart describing a method for treating epileptic seizures in a patient, using cortical lesioning, in accordance with an embodiment of the present specification.

### DETAILED DESCRIPTION

The present specification provides a system and a minimally invasive method for using cortical lesioning to treat epileptic seizures in a patient. In embodiments, multiple depth electrodes are inserted at predefined angles and depth in the vicinity of a suspected seizure focus through small burr holes drilled in the skull of the patient. Each depth electrode may have a plurality of EEG contacts, ranging from 10-20. In embodiments, an EEG image of the patient's brain enables determination of the location and suitability of an identified seizure focus for lesioning, whereafter lesions are generated and the seizure focus is ablated using heat. Thus, the systems and methods of the present specification allow for a single minimally invasive procedure for using cortical lesioning to treat epileptic seizures, as the depth electrodes inserted in the patient's brain for monitoring are also used for the ablation of seizure focus, eliminating the need for an additional surgical procedure.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It should also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

The term “EEG output” used in this disclosure include images, traces, charts, values, or other data indicative of an electroencephalographic analysis of brain electrical data.

In conventional systems, heat may be applied to areas of a patient's brain by inserting a laser optic fiber into a desired location in the brain and then using a power laser to heat the brain tissue via the optic fiber. Heat may be applied to areas of a patient's brain areas by transferring electrical energy through contacts positioned on the depth electrode, wherein an estimate of the energy needed for required ablative action may be computed beforehand. The temperature of the brain tissue may be measured by using a customized MRI procedure in order to control a size of the lesion generated in the patient's brain. However, since depth electrodes cannot be left in place within the brain during an MRI procedure, laser ablation with MRI monitoring is done after removal of the depth electrodes, and as a result it is not possible to monitor the EEG activity in the patient's brain during or after the ablative procedure. This precludes iterative ablation as well as electrographic assessment of the ablation. The feedback to the operator from observing ongoing brain activity is lost. In embodiments, and as described, the ablation is performed using the already embedded electrodes, and monitoring can be continued after ablation. After ablation, the heated, but not fully ablated, brain recovers over time, which may be one or more days, and may show residual seizure activity when monitored. A subsequent ablation can then be performed if indicated.

In various embodiments, the present specification provides a system that allows for lesioning of a patient's brain tissue while obtaining real time temperature of the tissue, EEG observation of the patient's brain during the lesioning process, and an iterative method for performing lesioning. Thus, in embodiments, a smaller degree of lesioning is performed initially, with subsequent additional lesioning if indicated by the EEG in order to preserve as much brain tissue as possible. In embodiments, the method of the present specification enables a surgeon to visualize the lesion on a 3D imaging system that shows the contact locations of the depth electrode in the brain, the MRI of the brain, and the generated lesion as a volume within the brain. In various embodiments, post-surgical MRI evaluation of the lesion in the patient's brain is fed into a database and artificial intelligence (AI) is used to improve the accuracy and degree of lesioning for providing improved outcomes of the lesioning/ablation procedure.

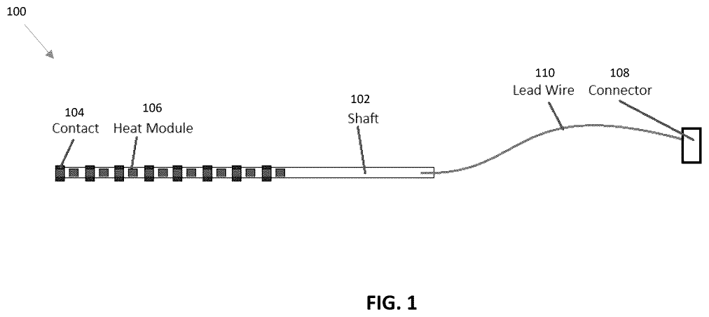

FIG. 1 illustrates a depth electrode comprising a plurality of contacts and heating modules, in accordance with an embodiment of the present specification. Depth electrode 100 comprises a shaft 102 coupled with one or more contacts 104 and one or more heating modules 106. The shaft 102 has a length defined by a longitudinal axis. In embodiments, the one or more contacts 104 and the one or more heating modules 106 are spaced apart along the longitudinal axis. The depth electrode 100 is insertable in a target area of a patient's brain such that at least one of the one or more contacts 104 is adapted to be used to monitor EEG activity of the patient's brain.

An optimal electrode placement allows for localization of the seizure focus. In embodiments, contact spacing ranges from 0.5 to 1 cm apart, with multiple electrodes dispersed in the area of interest. The optimal heat and sensing placement allow for both monitoring and control of the volume to be ablated. Energy added to the brain as heat at any specific location causes tissue heating based on elapsed time, rate of energy input, and thermal conductivity of adjacent tissue. The optimal number and placement of the heat modules will equal or exceed the number of contacts but is not limited by contact locations.

In an embodiment, the contacts 104 and the heating modules 106 are positioned on the shaft 102, as shown in FIG. 1. The positions of the contacts (typically between 4 and 16 contacts per electrode) are located to monitor the region of interest in the brain. The heat modules are also positioned within and around the region of interest where ablation is expected. The number of heat modules and their axial positions are set to provide heat generation and temperature measurements adequate for controlling the ablated volume. As shown by way of example only in FIG. 1, there are two heat modules per contact, including one that extends beyond the right-most contact. Shaft 102 is also coupled with a connector 108 at its distal end, via a lead wire 110. In embodiments, the contacts 104 are configured to enable recording of an EEG corresponding to a patient when the electrode 100 is inserted into the patient's brain, in order to obtain the location of the seizure focus. In embodiments, each heating module 106 is configured to measure a temperature of the surrounding brain area and generate heat within a predefined temperature range.

Cell death, which is the desired outcome, is time and temperature dependent, and is primarily due to protein denaturation. For most proteins, each 10 degrees C. (18 degrees F.) increase in temperature results in a 10-fold increase in denaturation. In some embodiments, the predefined temperature range, in which heat is generated, is between 150 and 170 degrees F. In some embodiments, a temperature of 160 degrees F., for a duration on the order of a few seconds, is considered an endpoint for thermal tissue death. Tissue death is both time and temperature dependent, however, and with the controlled heat generation, the temperature sensing and temperature modeling used a time/temperature paradigm allows better control of the ablated volume.

In embodiments, the shaft 102 is made of materials that can withstand a predefined temperature range (heat tolerant) generated during lesioning of the patient's brain tissue. In the embodiment shown for illustration, depth electrode 100 comprises eight (8) contacts 104 and eight (8) heat modules 106 positioned on the heat tolerant shaft 102. Other embodiments of existing depth electrodes have 4 to more than 20 contacts, and other electrode configurations including grids, could utilize the heat modules. The electrode, as shown in FIG. 1, terminates in an identified (ID'd) connector 108. The connector has a digital ID that is read and tracked by the system to verify that the correct set of heating elements are activated for lesioning. The connector ID is a guaranteed unique digital code to track each electrode and ensures that the correct electrode(s) is attached to an EEG recorder and heating system, collectively an EEG controller.

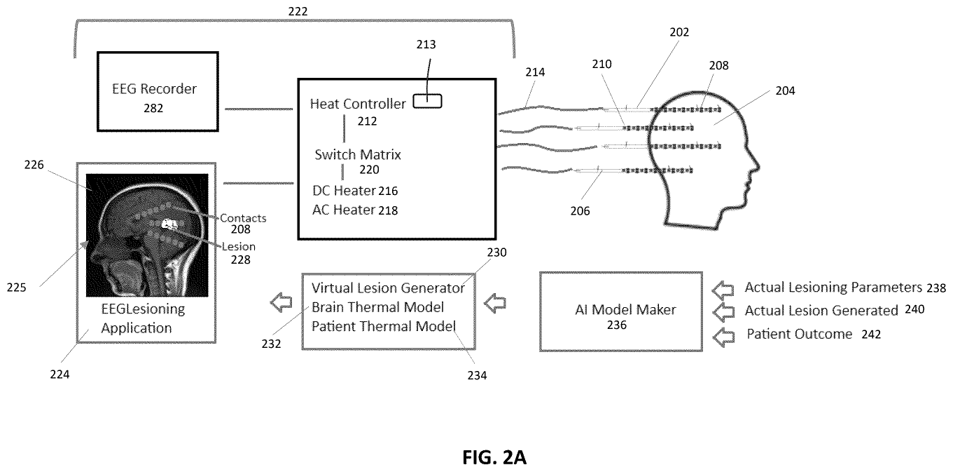

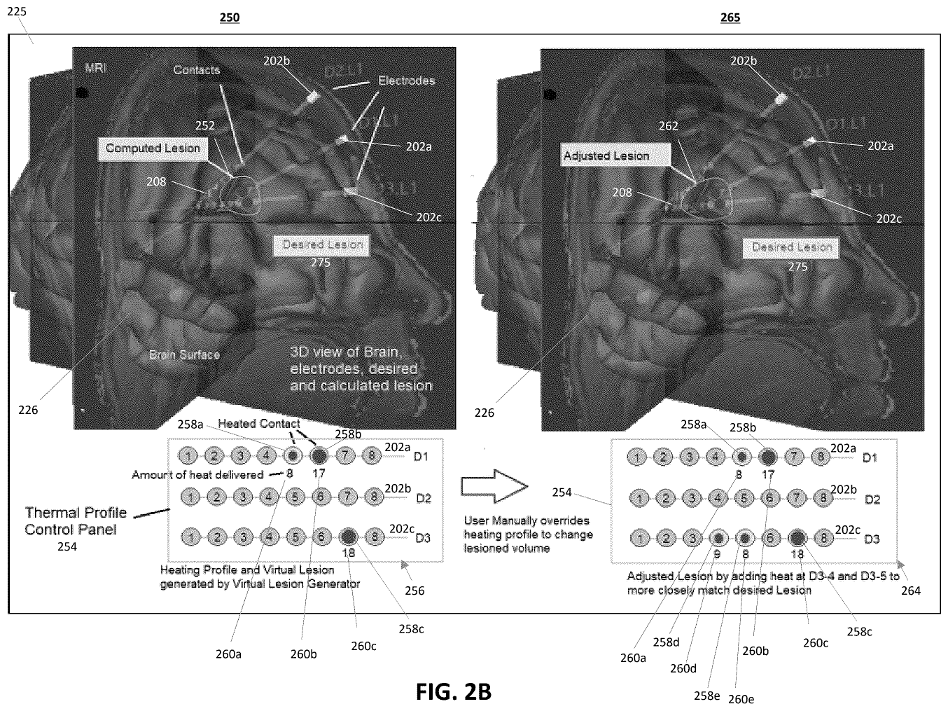

In an embodiment, the heating modules 106 are coupled with at least one heat controller (shown in FIG. 2) via the connector 108, that is configured to enable concurrent monitoring of the patient's EEG, measurement of the temperature of predefined portion of the patient's brain and application of heat to the predefined portion of the patient's brain comprising the seizure focus. Thus, the heat controller is configured to measure the temperature of a predefined portion of the patient's brain and is also configured to vary the electrical heating supplied to the predefined portion. In some embodiments, the connector 108 is configured such that it allows switching the contacts 104 from EEG inputs to the heating system.

The heat controller, a switch matrix and an EEG amplifier are all controlled by an EEG lesioning software. Command and control may use, but is not limited to, serial protocols such as ethernet, I2C, CAN, USB, or proprietary time synchronization modules. A heating module is activated for heating and sensing using a serial protocol. Electrical energy for the heating may be carried by additional wires running to the electrode(s) and controlled by the heating module. Each subsystem used within the cortical lesioning system of the present specification may include redundant enable, abort, and fail-safe features to mitigate inadvertent power delivery. In an embodiment, where fractional second tissue death in the patient's brain is assumed, the portion of the patient's brain comprising the seizure focus is heated up to a temperature of 160 degrees F. In other embodiments, the portion of the patient's brain comprising the seizure focus may be heated to different temperature ranges depending upon the patient's individual condition.

In some embodiments, the heat controller is an electrical unit that is configured to deliver electrical power to at least one of the one or more contacts 104 or the one or more heating modules 106. The heat modules 106 change electrical energy to thermal energy by passing current through a resistor. Power is current x current x resistance, that is P=I²R where P is power, I is current and R is resistance. The power can be controlled by changing the drive current. Heating between contacts 104 also uses electrical energy, delivered at high frequency (e.g. 100 kHz) and the brain tissue itself provides the resistance.

In alternate embodiments, the heat controller is configured to deliver other forms of energy such as, but not limited to, radiofrequency (RF) energy to at least one of the contacts 104 or heating modules 106.

In embodiments, the contacts 104 are spaced 5 mm apart on the shaft 102 in order to provide a desired resolution of the brain activity via EEG recording. In other embodiments the contact spacing may range from 2.5 to 10 mm apart, and the contacts 104 may or may not be uniformly spaced along the electrode. In embodiments, the heat modules 106 may be spaced at a distance less than 5 mm apart on the shaft 102 in order to provide a desired resolution for the lesioning process. In an embodiment, the heating modules 106 are not configured to measure, monitor, or detect the brain's electrical activity and transfer signals indicative of the brain's electrical activity to a monitor. In an embodiment, the heating modules 106 are configured such that they can provide heat to as well as measure the temperature of the patient's brain. In embodiments, some or all of the heating modules 106 are configured to measure the temperature of the patient's brain and are integrated with the contacts 104. In embodiments, some or all of the heating modules 106 are configured to generate heat from an applied electrical current and are integrated with the contacts 104. In an embodiment, the heat controller may be configured to enable either the contacts 104 or the heating modules 106 to apply heat to the predefined portion of the patient's brain.

FIG. 2A is a diagrammatic representation of a system for using cortical lesioning to treat epileptic seizures, in accordance with an embodiment of the present specification. System 200 comprises depth electrodes 202 which are inserted into a patient's brain 204, wherein each depth electrode 202 comprises a shaft 206 coupled with a plurality of contacts 208 and a plurality of heating modules 210, as also described with respect to FIG. 1 above. Each shaft 206 is coupled to a heat controller 212, via a lead wire 214. In an embodiment, the heating modules 210 are passive elements such as, but not limited to an NTC thermistor, while in other embodiments, the heating modules 210 comprise a semiconductor chip with temperature measurement, addressable heat control, and heat generation capability.

In embodiments, heat controller 212 includes both a DC heater 216 and an AC heater 218 which are toggled via a switch matrix 220. The switch matrix 220 is configured to couple at least one of the one or more contacts and/or the one or more heating modules to at least one of an AC power source or a DC power source. Heat controller 212 is also coupled to an EEG controller 222 which may comprise an EEG recorder 282 and, preferably and optionally, an EEG lesioning software application 224.

In embodiments, both the EEG lesioning software application is part of, along with a lesion viewer 225, an EEG controller 222 that is configured to capture and analyze brain wave activity, using an EEG recorder 282, and cause the analyzed brain wave activity, in the form of data, charts, traces, tables, or other visual indices, to be displayed on an EEG monitor. In embodiments, the EEG lesioning software application 224 is executed on a computing device. The EEG lesioning software application 224 includes a plurality of instructions of programmatic code which, when implemented, cause the computing device to perform a plurality of functions to enable lesioning of one or more areas of the patient's brain. In embodiments, the EEG lesioning software application 224 is configured to generate the lesion viewer 225, which is, in embodiments, a graphical user interface (GUI), which is used by a surgeon to specify, observe, and adjust the lesioning desired.

In embodiments, the EEG lesioning software application 224 is configured to receive input from a virtual lesion generator 230, a brain thermal model 232 and a patient thermal model 234 which, in turn, are generated by an AI based model maker 236 that receives as input a patient's actual lesioning parameters 238, data 240 corresponding to the actual lesions generated in the patient's brain and the patient outcome 242. In some embodiments, the virtual lesion generator 230 and the AI based model maker 236 are modules implemented on the computing device that includes the EEG lesioning software application 224. In embodiments, the virtual lesion generator 230 and the AI based model maker 236 include a plurality of instructions of programmatic code which when implemented cause the computing device to enable related functionalities. In some embodiments, the EEG lesioning software application 224, the virtual lesion generator 230 and the AI based model maker 236 are separate modules in data communication with each other. Alternatively, in some embodiments, the EEG lesioning software application 224 may include the programmatic code for the virtual lesion generator 230 and the AI based model maker 236.

In embodiments, the heat controller 212 supports concurrent monitoring of the continuous EEG of the patient's brain 204 as well as heating a desired portion of the brain and temperature measurement of the brain. In some embodiments, the EEG lesioning software application 224 may be configured to cause the heat controller 212 to deliver heat to one or more of the contacts 208. In an embodiment, heat is applied to brain tissue via the contacts 208 which are made of conductive materials such as, but not limited to platinum/iridium or stainless steel and are placed in contact with the brain tissue in order to pick up EEG electrical activity of the brain. In some embodiments, the EEG lesioning software application 224 may be configured to cause the heat controller 212 to deliver heat to one or more of the heating modules 210. In a preferred embodiment, the heating modules 210 provide controlled heating of the patient's brain, independent of factors such as, but not limited to tissue impedance, thereby eliminating the need for a complex switch, a second high frequency power source, and current flow through the brain. Heating the patient's brain 204 by using the heating modules 210 requires the use of DC power provided by DC heater 216, while heating the patient's brain 204 by using the contacts 208 requires the use of AC power at a high frequency provided by the AC heater 218.

In embodiments, the lesion viewer or GUI 225, generated by the EEG lesioning software 224, provides a virtual model 226 of the depth electrodes 202 which is generated from trajectory information used to place the depth electrodes 202 into the patient's brain 204. The virtual model 226, viewable by a user/surgeon via the lesion viewer 225, allows a user/surgeon to see an MRI image of the patient's brain 204, a cortical surface presentation of the brain, the proposed lesion 228, and the actual temperatures in the brain in a 3D volumetric image. The EEG lesioning software application 224 is configured to identify and allow for the locations of the contacts 208 and heating modules 210/temperature sensors placed in the brain to be further aligned after surgery using CT imaging to obtain a more precise virtual model 226 of the brain. In embodiments, a surgeon may use the virtual model 226 to visualize the brain volume to carry out lesioning.

As shown in FIG. 2A, the surgeon describes a volume 228 (that is, the proposed lesion 228) of cortical tissue to be ablated. Heat will move from each heat source outward more or less rapidly depending on proximity to blood vessels, proximity to ventricles, and so forth. The three-dimensional heating will always be highest close to the heating element, but the temperature distribution can be calculated using known thermal characteristics of the brain (brain thermal model), the actual geometry of the patient's brain obtained from the MRI (patient thermal model), and can be controlled by changing the rate of heating and the duration of heating. The volume 228 of ablation can be changed to best match the desired volume by adjusting which heating elements are activated at what power level and for what duration. A virtual lesion generator calculates the best thermal protocol and shows the resulting lesion virtually in the 3D brain model. Manual adjustments to the protocol will change the resulting lesion, and allow overriding the virtual lesion protocol.

The virtual lesion generator 230 computes the expected 3D thermal profile in the brain over time as heat is added in differing amounts at different electrodes over time. The expected 3D shape of the guaranteed ablated volume, and the 3D shape of guaranteed preserved volume are modified by the heating sequence. A sequence can be tailored to optimize ablation and preservation. The brain thermal model 232 describes how heat will flow and dissipate and could use the patients MRI as well as known blood flow, location of ventricles and other non-uniform thermal characteristics to create the model. A good model for heat input and heat spread would allow consideration of adjunctive paradigms such as whole body cooling or pharmaceutical control of blood flow to further optimize the lesioning process.

In embodiments, the brain thermal model 232 is generated and used to predict a spread of heat within the patient's brain 204 and which is, in turn, used to determine an application pattern of heat in order to achieve a desired temperature at a desired location in the brain. In some embodiments, a generic model (or a patient specific model based on the patient's actual MRI images) is used to control the heating sequence of the patient's brain 204. However, factors such as, but not limited to, individual brain characteristics and blood flow within the brain may impact the actual temperatures reached while heating the brain. In some embodiments, the present specification provides an improved heating protocol for the identified areas of the patient's brain 204. In an embodiment, the EEG lesioning software application 224 and the heat controller 212 are configured to determine the thermal characteristics of the patient's brain 204 by minimally heating (e.g. to 105 degrees F., well below lesioning temperatures) contacts 208 or heating modules 210 and measuring the spread of heat and the decay rate of the heat within the patient's brain 204. The measured values are used to establish a thermal baseline to verify and adjust the computed thermal model 232. Thus, the minimal heating temperature, of, for example, 105 degrees F., is far below temperatures that can cause tissue damage, but affords a finer calibration of the actual thermal model of the patient's brain than can be estimated from MRI alone.

The seizure focus is determined using EEG, by observing actual seizures progress electrically, by stimulating contacts in and around the suspected seizure focus, as well as clinical information. This determination would often be done by the neurologist. The volume 228 to ablate is chosen to envelope the seizure focus and minimize damage to unaffected brain tissue. The volume 228 could be computed algorithmically or entered manually into the 3D brain image, with care to avoid critical structures.

In embodiments, the virtual lesion generator 230 is configured to compute a lesioning sequence for generating a lesion approximating the lesion desired. The EEG lesioning software application 224 is configured to modify the sequence iteratively to expand or contract the generated lesion and may present a modified lesioning sequence based on actual patient measurements for verification or adjustment by the surgeon. Since lesioning the patient's brain is an irreversible process, in various embodiments, the present specification provides a lesioning sequence enabling monitored lesioning, wherein the temperature gradient in the brain is monitored and heating may be halted when the brain's temperature reaches a predefined/desired threshold, whereafter a user may be allowed to proceed with additional lesioning as desired. Thus, in embodiments, a generated lesion along with corresponding monitored temperature, in certain cases, primarily due to blood flow in the patient's brain, does not progress at a predefined/computed rate. In an embodiment, the EEG lesioning software application 224 comprises a seizure inhibit stimulator for stopping any seizure activity occurring in the patient's brain 204 during the lesioning procedure. In embodiments, the patient's brain 204 is stimulated for observing the brain's reaction after insertion of depth electrodes 202 into the brain, before providing heat to the brain via the electrodes 202. If the stimulation inadvertently causes a seizure in the brain, the seizure may be terminated by applying a known standard sequence of stimulations to the brain.

Thus, the EEG lesioning software application 224 is configured to cause the heat controller 212 to generate electrical current based upon a lesioning sequence that defines a pattern for the one or more lesions. In embodiments, the EEG lesioning software application 224 generates the lesioning sequence and communicates the lesioning sequence to the heat controller 212.

In an embodiment, a radio frequency (RF) cautery unit is used as a heating element and is coupled with the lead wires 214 of the contacts 208 to heat the brain tissue. The use of RF (or alternating current (AC) at a high frequency) prevents unwanted stimulation of the brain. In other embodiments, an AC supply cautery unit is coupled with the lead wires 214 of the contacts 208 to heat the brain tissue. In this configuration, the power delivered via the AC supply may be monitored continuously, which is not always possible while using an external cautery unit. Conventionally, coupling an external cautery unit with a system such as system 200 requires decoupling the EEG controller 222 from at least one or more of the contacts 208. In addition, an external cautery unit itself generates noise that precludes monitoring of EEG activity within the brain, while the cautery unit is activated, thereby preventing monitoring of the EEG during the ablation procedure. Thus, in some embodiments, the use of the heat controller 212 and switch matrix 220 allow concurrent heating and monitoring of the brain, which increases the efficiency/accuracy of the ablation procedure.

In embodiments, the heating modules 210 are not in electrical contact with the patient's brain 204, however, are in thermal contact with the patient's brain 204 through shafts 206 of the depth electrodes 202. Heat controller 212 enables precise and controlled heating of the heating modules 210 by using the DC heater 216, the AC heater 218 and the switch matrix 220. Since the heating modules 210 do not monitor/pick up EEG activity of the brain 204, the modules do not generate any electrical artifact, thereby enabling heating, temperature monitoring, and EEG monitoring of the brain 204 to be performed concurrently and simultaneously. In embodiments, the heat controller 212 is enabled to switch between heating, temperature measurement and EEG recording of the brain 204 either concurrently or on a time multiplexed basis. In embodiments, time multiplexing is used when the electrical activity of heating or measuring the temperature of the brain 204 interferes with the signal generated from the measurement of the brain using EEG, which is several orders of magnitude smaller than the electrical activity of heating the brain. In embodiments, a frequency of time multiplexing (switching between heating, temperature measurement and EEG recording of the brain 204) is dependent upon a recovery time of an EEG amplifier employed in the system for obtaining EEG recording of the brain and may range from a few milliseconds to several seconds.

In various embodiments, the controller 222, which includes EEG lesioning software application 224 is configured to capture and cause lesion viewer 225 to generate an EEG output using data from at least one depth electrode: a) while the heat controller 212 is providing electrical current to at least one of the one or more contacts or the one or more heating modules or b) only when the one heat controller 212 is not providing the electrical current to at least one of the one or more contacts or the one or more heating modules. Stated differently, in some embodiments, the one or more contacts are configured to only monitor electrical activity of the patient's brain and are not configured to receive the electrical current from the heat controller 212 to generate heat. In some embodiments, the one or more contacts are configured to monitor electrical activity of the patient's brain and to receive the electrical current from the heat controller 212 to generate heat.

In some embodiments, the one or more contacts are configured to only monitor electrical activity of the patient's brain and are not configured to receive the electrical current from the heat controller 212 to generate heat while the one or more heat modules are configured to receive the electrical current from the heat controller 212 to generate heat.

In an embodiment, the heat controller 212 comprises a manual actuator 213 enabling connection with electrical power wherein, the actuator carries power and is not merely a software input to the heat controller 212. Hence, in embodiments, the present specification provides a safety feature in the manner of a manually controlled actuator which can be used to control the power/heat being delivered to the patient's brain 204 during operation of the system 200, in times such as, but not limited to a system software or firmware failure preventing inadvertent/uncontrolled heating of the brain via the heat controller 212. The EEG lesioning software application 224, with the manual actuator enabled, is configured to control the heat controller 212, monitor the actual temperatures of the patient's brain 204, and generate iterative views of the status of the lesioning of the brain. In embodiments, the heat controller 212 is connected only to the depth electrodes 202 that are required for lesioning the patient's brain 204, wherein the lead wires 214 of the electrodes 202 are routed through the heat controller 212 to the EEG controller 222.

FIG. 2B are pictorial virtual representations of a computed lesion and an adjusted lesion generated by the virtual lesion generator 230 for viewing in the lesion viewer 225, in accordance with some embodiments of the present specification. The lesion viewer 225 is configured to display the virtual model 226 of the patient's brain including virtual representations of first depth electrode 202a, second depth electrode 202b, and third depth electrode 202c, and locations of the associated contacts 208. A first view 250 shows a computed lesion 252 determined by the virtual lesion generator 230. A thermal profile control panel 254 shows a first heating profile or pattern 256 of the contacts 208, as computed by the virtual lesion generator 230. For example, the first heating profile or pattern 256 corresponds to providing heat at a 5th contact 258a and at a 6th contact 258b of the first depth electrode 202a, providing no heat at any of the contacts of the second depth electrode 202b and providing heat at a 7th contact 258c of the third depth electrode 202c. Thus, the computed lesion 252 corresponds to the first heating profile or pattern 256. In some embodiments, the amount of heat delivered is computed and displayed (in terms of a predefined heat unit) in association with each of the heated contacts. For example, the contact 258a has an associated first heat value 260a, the contact 258b has an associated second heat value 260b and the contact 258c has an associated third heat value 260c.

The thermal profile control panel 254 supports the user in modulating (expand or contract) the lesioned volume corresponding to the computed lesion 252, by manually overriding the first heating profile or pattern 256 in order to generate a second heating profile or pattern 264 corresponding to an adjusted lesion 262. A second view 265 shows the adjusted lesion 262 based on the second heating profile or pattern 264 of the contacts 208 based on the user's manual adjustment of the first heating profile or pattern 256. In the second view 265, the thermal profile control panel 254 shows the second heating profile or pattern 264. For example, the second heating profile or pattern 264 corresponds to providing heat at the 5th contact 258a and at the 6th contact 258b of the first depth electrode 202a, providing no heat at any of the contacts of the second depth electrode 202b, providing heat at the 7th contact 258c of the third depth electrode 202c and additionally providing heat at a 4th contact 258d and at a 5th contact 258e of the third depth electrode 202c in order to more closely match the desired lesion 275. Thus, the adjusted lesion 262 corresponds to the second heating profile or pattern 264. In this example, the adjusted lesion 262 represents an expanded lesion volume compared to that of the computed lesion 252. Also shown is a fourth heat value 260d associated with the contact 258d and a fifth heat value 260e associated with the contact 258e.

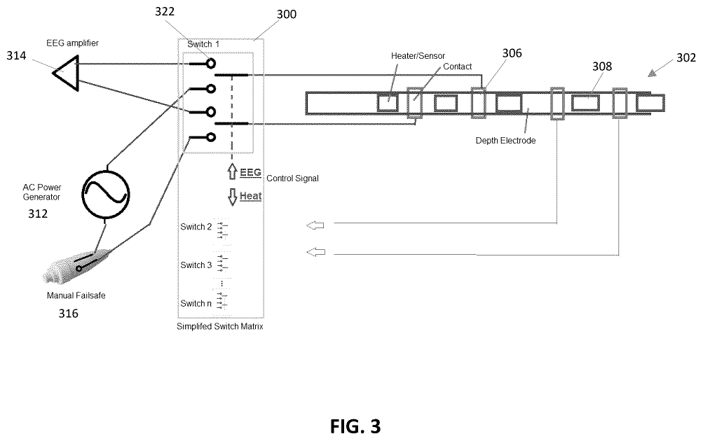

FIG. 3 is a diagrammatic representation of a switch matrix for use in a system using cortical lesioning to treat epileptic seizures, in accordance with an embodiment of the present specification. In embodiments, a switch matrix 300 is coupled with each depth electrode 302. Each depth electrode 302 may be inserted in a patient's brain (brain 204 in FIG. 2A) for isolating EEG signals being monitored via contacts 306 positioned on the electrode 302 and also for heat generation/temperature measurement being conducted by heating modules 308 positioned on the electrode 302. Switch matrix 300 comprises a plurality of electrical switches 322, wherein at least one electrical switch 322 is coupled with at least one of each of the contacts 306 for connecting each contact 306 with either an AC power source 312 or an EEG amplifier 314. In embodiments, the switches 322 are one of a semiconductor relay or a mechanical relay. In some embodiments, each of the contacts 306 is also coupled with a manual switch 316 for connecting and disconnecting the contacts 306 from a heating source, if needed, in order to avoid damaging the contacts 306 that are being used to monitor EEG activity in the brain, or in order to shunt electrical power through the EEG monitoring contacts 306.

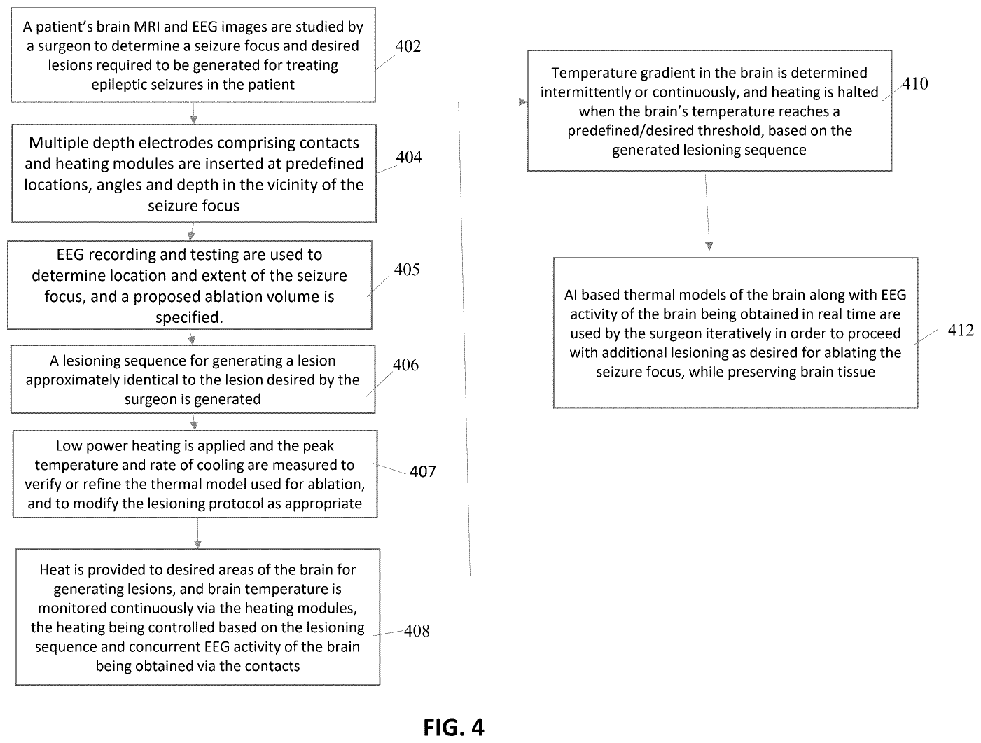

FIG. 4 is a flowchart illustrating a method of treating epileptic seizures in a patient, using cortical lesioning, in accordance with an embodiment of the present specification. At step 402, a patient's brain MRI and EEG images are studied by a surgeon for determining a seizure focus and lesions that may be required to be generated for treating epileptic seizures in the patient.

At step 404, multiple depth electrodes, comprising contacts and heating modules, are inserted at predetermined locations, angles and depths in the vicinity of a seizure focus within the brain. In some embodiments, the predetermined depths may range from 2 to 15 cm. The electrode placement and entry points are chosen to locate the contacts in the vicinity of the seizure focus, while avoiding trauma to blood vessels and uninvolved brain. The insertions are through small burr holes drilled in the skull of the patient. In embodiments each of the depth electrodes comprise a shaft coupled with a plurality of contacts and a plurality of heating modules.

At step 405, the EEG recording and the patient are observed and tested until the seizure focus can be anatomically localized in the context of the monitoring electrodes. A volume for ablation is generated which will encompass the seizure focus. Stated differently, the EEG recording and testing are used to determine location and extent of the seizure focus, and a proposed ablation volume is specified.

At step 406, a proposed lesioning sequence is generated to ablate the specified volume using a brain thermal model based on known brain thermal characteristics and on the patient's specific anatomy determined from the MRI. Thus, a lesioning sequence for generating a lesion approximately identical to the lesion desired by the surgeon, is generated.

At step 407, low power heating is applied and the peak temperature and rate of cooling are measured to verify or refine the brain thermal model used for ablation, and to modify the lesioning protocol as appropriate.

At step 408, heat is provided to desired areas of the brain in order to generate lesions, and temperature of the brain is monitored continuously via the heating modules, the heating being controlled based on the lesioning sequence and concurrent EEG activity of the brain being obtained via the contacts.

At step 410, a temperature gradient in the brain is determined continuously, and heating is halted when the brain's temperature reaches a predefined/desired threshold, based on the generated lesioning sequence.

At step 412, AI based thermal models of the brain along with EEG activity of the brain, being obtained in real-time, are used by the surgeon iteratively in order to proceed with additional lesioning as desired for ablating the seizure focus, while preserving brain tissue.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A cortical lesioning system comprising:
at least one depth electrode comprising
a shaft having a length defined by a longitudinal axis;
one or more contacts positioned on the shaft and spaced apart along said longitudinal axis; and
one or more heating modules positioned on the shaft and spaced apart along said longitudinal axis, wherein the depth electrode is configured to be insertable in a target area of a patient's brain and wherein at least one of the one or more contacts is adapted to be used to monitor EEG activity of the patient's brain;
at least one heat controller coupled with the shaft for providing an electrical current to at least one of the one or more contacts or of the one or more heating modules for generating one or more lesions in the target area of the patient's brain; and
an EEG recorder configured to capture and generate an EEG output using data from said at least one depth electrode, wherein said EEG recorder is configured to capture and generate said EEG output a) while the at least one heat controller is providing said electrical current to the at least one of the one or more contacts or of the one or more heating modules or b) only when the at least one heat controller is not providing said electrical current to the at least one of the one or more contacts or of the one or more heating modules.

2. The system of claim 1, wherein the at least one depth electrode is adapted to be inserted at a predefined angle and depth in the vicinity of a seizure focus in the patient's brain.

3. The system of claim 1, wherein the heat controller is further configured to generate said electrical current based upon a lesioning sequence, and wherein said lesioning sequence defines a pattern for the one or more lesions.

4. The system of claim 3, further comprising an EEG controller, wherein the EEG controller comprises the EEG recorder and an EEG lesioning software application, wherein, when executed, the EEG lesioning software application generates said lesioning sequence and communicates the lesioning sequence to the heat controller.

5. The system of claim 4, wherein the EEG controller further comprises the heat controller.

6. The system of claim 1, wherein the heat controller comprises a switch matrix configured to couple at least one of the one or more contacts or the one or more heating modules to at least one of an AC power source or a DC power source.

7. The system of claim 1, wherein the shaft is coupled to the heat controller via a lead wire.

8. The system of claim 4, wherein, when executed, the EEG lesioning software application is further adapted to generate a virtual model of the at least one depth electrode and wherein the EEG lesioning software application is configured to generate the virtual model by using trajectory information indicative of a placement of the at least one depth electrode in the target area of the patient's brain.

9. The system of claim 8, wherein the virtual model is configured to depict a 3D volumetric image comprising data representative of at least one of the patient's brain, a cortical surface of the brain, the one or more lesions, and an actual temperature in the brain.

10. The system of claim 1, wherein the one or more contacts are spaced along the longitudinal axis such that adjacent ones of the one or more contacts are separated from each other by a distance in a range of 2.5 mm to 10 mm.

11. The system of claim 10, wherein the one or more contacts are configured to only monitor electrical activity of the patient's brain and are not configured to receive the electrical current from the heat controller to generate heat.

12. The system of claim 10, wherein the one or more contacts are configured to monitor electrical activity of the patient's brain and to receive the electrical current from the heat controller to generate heat.

13. The system of claim 1, wherein the one or more heat modules are spaced along the longitudinal axis such that adjacent ones of the one or more heat modules are separated from each other by a distance less than 5 mm.

14. The system of claim 13, wherein the one or more contacts are configured to only monitor electrical activity of the patient's brain and are not configured to receive the electrical current from the heat controller to generate heat while the one or more heat modules are configured to receive the electrical current from the heat controller to generate heat.

15. A method of lesioning portions of a patient's brain, comprising:
providing at least one depth electrode, wherein the at least one depth electrode comprises a shaft having a length defined by a longitudinal axis, one or more contacts positioned on the shaft and spaced apart along said longitudinal axis, and one or more heating modules positioned on the shaft and spaced apart along said longitudinal axis, wherein the depth electrode is configured to be insertable in a target area of a patient's brain and wherein at least one of the one or more contacts is adapted to be used to monitor EEG activity of the patient's brain;
inserting the at least one depth electrode in a target area of the patient's brain;
generating at least one lesion in the target area of the patient's brain by providing an electrical current to at least one of the one or more contacts or of the one or more heating modules in order to generate heat from the at least one of the one or more contacts or of the one or more heating modules; and
generating an EEG output using data from said at least one depth electrode, wherein said EEG output is generated a) said electrical current is being provided to the at least one of the one or more contacts or of the one or more heating modules or b) only when said electrical current is not being provided to the at least one of the one or more contacts or of the one or more heating modules.

16. The method of claim 15, further comprising inserting the at least one depth electrode at a predefined angle and depth in the vicinity of a seizure focus in the patient's brain.

17. The method of claim 15, further comprising generating the electrical current based on a lesioning sequence generated by a lesioning software application.

18. The method of claim 16, further comprising heating the seizure focus to a temperature of 160 degrees F.

19. The method of claim 15, further comprising using data indicative of a trajectory of placement of the at least one depth electrode in the target area of the patient's brain to generate a virtual model of the at least one depth electrode.

20. The method of claim 19, further comprising generating a 3D volumetric image of the patient's brain, a cortical surface presentation of the brain, the one or more lesions, and a temperature in the brain.

21. The method of claim 15, wherein the one or more contacts are spaced along the longitudinal axis such that adjacent ones of the one or more contacts are separated from each other by a distance in a range of 2.5 mm to 10 mm.

22. The method of claim 21, wherein the one or more contacts are configured to only monitor electrical activity of the patient's brain and are not configured to receive the electrical current to generate heat.

23. The method of claim 21, wherein the one or more contacts are configured to monitor electrical activity of the patient's brain and to receive the electrical current to generate heat.

24. The method of claim 1, wherein the one or more heat modules are spaced along the longitudinal axis such that adjacent ones of the one or more heat modules are separated from each other by a distance less than 5 mm.

25. The method of claim 24, wherein the one or more contacts are configured to only monitor electrical activity of the patient's brain and are not configured to receive the electrical current to generate heat while the one or more heat modules are configured to receive the electrical current to generate heat.
